(19) United States
(12) Patent Application Publication
(10) Pub. No.: US 2013/0123585 A1
(43) Pub. Date: May 16, 2013

(54) PORTABLE BRAINWAVE MEASURING AND CONTROLLING SYSTEM

(71) Applicant: Sungchul Kang, Busan (KR)

(72) Inventor: Sungchul Kang, Busan (KR)

(21) Appl. No.: 13/812,718

(22) PCT Filed: Jul. 28, 2011
(86) PCT No.: PCT/KR2011/005551

§ 371 Date: Jan. 28, 2013

(30) Foreign Application Priority Data
Jul. 28, 2010 (KR) 10-2010-0072676

(51) Int. Cl. A61B 5/0478; A61B 5/04; A61B 5/11; A61B 5/00

(52) U.S. Cl. CPC A61B 5/0478; A61B 5/7203; A61B 5/04014; A61B 5/742; A61B 5/6814; A61B 5/0006; A61B 5/11
USPC 600/301

(57) ABSTRACT

The present invention relates to a portable brainwave measuring method, which measures a weak brainwave signal detected from a human scalp through a noninvasive method to measure a degree of concentration through an analysis and process of the detected brainwave signal, and a control method enabled for short distance control of an electronic device or remote monitoring through the internet, by using the brainwave signal. An acceleration sensor is put on the head of a user, and a brainwave detecting means put on the head of a user to detect a brainwave and the acceleration sensor that outputs an acceleration value of three axes including XYZ axes as a signal of predetermined data are used to detect movement of the head, to thereby control the direction and speed of a brainwave-related device. In more detail, after a signal outputted from the brainwave detecting means is converted into a wireless signal and transmitted, a value of the wireless signal is inputted to a receiving unit of a display device and is expressed numerically. By setting the value inputted to the display device, a portable brainwave measuring device provides accurate device control. A control system is characterized in that a signal according to the slope direction of the head is detected and analyzed, and then numerically expressed by using 6 brainwave signals such as a delta wave (δ), theta wave (θ), alpha wave (α), SMR wave, beta wave (β) and gamma wave (σ), which are measured through the portable brainwave measuring device and the acceleration sensor put on the head. A wireless system, including a short distance wireless module for transmitting/receiving the wireless signal, includes a wireless receiving module, a signal analyzing unit, and a control output unit. Additionally, the wireless system is connected to a PC or controls a short/long distance external device.

### CROSS-REFERENCE TO RELATED APPLICATION

This application claims the benefit of Korean Patent Application No. 10-2010-0072676, filed on Jul. 28, 2010 in the Korean Intellectual Property Office, the disclosure of which is incorporated herein by reference.

### TECHNICAL FIELD

The present invention relates to an apparatus and method for controlling a brainwave-related external instrument in such a way to display a signal wirelessly received via a local area wireless module from an apparatus measuring a brainwave and an apparatus measuring an acceleration value in a 3-axis direction and to use the signal, and in particular to a portable brainwave measuring apparatus having features in that six brainwave signals (delta wave δ, theta wave θ, alpha wave α, SMR wave, beta wave β and gamma wave σ) analyzed by a micro controller unit (MCU) by detecting a brainwave from a user can be easily checked on a visual apparatus by a user, and a brainwave-related external instrument control system using the values measured by an apparatus configured to measure the brainwaves and by an apparatus configured to measure an acceleration value of a 3-axis direction.

### BACKGROUND ART

Generally speaking, a brainwave is a kind of wavelengths with a potential difference of tens of micro volts and a frequency of below 30 Hz which are measured from a head skin of a human and is a physical value reflecting a consciousness state of a human. Depending on the frequencies, the brainwave can be classified into six kinds: delta wave δ, theta wave θ, alpha wave α, SMR wave, beta wave β, and gamma wave σ.

The delta wave is a kind of brainwaves with a frequency of 0.5 to 3.5 Hz, which is directly related to a non-conscious state or a sleeping state. The theta wave is a kind of brainwaves with a frequency of 4 to 7 Hz, which is relates to when a person lightly sleeps or dozes. The alpha wave is a kind of brainwaves with a frequency of 8 to 11 Hz, which is related to a stable and comfort state. The SMR wave is a kind of brainwaves with a frequency of 12 to 15 Hz, which is related with when a person intensively focuses on something, and the beta wave is a kind of brainwaves with a frequency of 16 to 30 Hz, which is related to when a person is slightly nervous and focuses while he acts with eyes open. The gamma wave σ is a kind of brainwaves with a frequency of 30 to 50 Hz, which is related to when a person thinks of something deeply or focuses on a high dimensional logic.

In recent years, many researches are being conducted on the controls of a thing by using brainwaves. More than 20 to 30 medical brainwave measurement instruments are being developed with the aid of a brainwave measurement electrode. By installing a certain program on a personal computer, a user plays a game on the personal computer in which a graphically processed vehicle runs with the aid of a concentration of consciousness or an arrow can be made to fly. Only a small number of brainwave electrodes might be installed for thereby detecting a brainwave signal, so a ping pong ball might be controlled to move upward by rotating a pen or a toy vehicle can be controlled to run forwards, which are all restricted to a simple on-off operation-based instrument.

The conventional brainwave measurement and control apparatus has a complicated measurement method when a user outputs a specific brainwave along with some difficulties in terms of a visual check. A result of the measurement can be checked only when it is connected to a specific process system such as a brainwave analysis program or a personal computer, not by way of a specifically processed numeric value. Therefore, it is hard for an ordinary person to have systematic spirit concentration power training. In case of a brainwave-related instrument control method using a brainwave, its application is very limited. Only a simple control system might be adapted, and a compatibility with other brainwave-related instruments is not good.

### DISCLOSURE OF INVENTION

Accordingly, the present invention is made to improve the above-mentioned problems, and it is an object of the present invention to provide a portable brainwave measuring and controlling system which has features in that a signal is detected by a brainwave detection part which is worn on a user's head skin and is processed in a numeric form, so the signal related to a brainwave can be visually displayed, a result of which is feed back for the sake of specific training on concentrations and an efficient operation, and a numerically processed value on a brainwave can be set, so a brainwave-related external instrument can be accurately controlled.

To achieve the above objects, the portable brainwave measuring and controlling system according to the present invention has features in that there are provided a display part which is formed of a brainwave detection part worn on a user's head for the purpose of detecting brainwaves and an acceleration sensor, so the brainwaves detected by the brainwave detection part is classified into six brainwave signals depending on the frequencies for thereby turning on and off LEDS corresponding to each signal; a display part outputting a changed value of a signal inputted from a receiving part of the portable brainwave measuring and controlling system; and a storing part comparing and storing the brainwave signal itself and the resultant values analyzed over the process of the MCU part and having a function of checking the states of the values. In addition, there is further provided a system which makes it possible to control an external instrument related with a brainwave in various ways with the aid of a wireless communication module such as a personal computer or a blue tooth by using a data numerically processed by an acceleration sensor and a brainwave detection part, while making sure that one or multiple users can measure spirit concentrations by a time division wireless communication method and an objective check of a comparative analysis can be possible through a numerical check.

The portable brainwave measuring and controlling system comprises a data transmission system 100 and a data receiving system 200. As shown in FIG. 1, the data transmission system 100 has features in that a brainwave signal is transferred to an Electro Encephalo Graph (EEG) input part 104 through three brainwave detection headset sensors 102, and a brainwave signal received from the EEG input part 104 and the data measured by the acceleration sensor 120 are transferred to an amplification and filter part 106 for the sake of noise filtering, and a brainwave length is amplified and inputted into the MCU 108 for the sake of process. At this time, the brainwave signal is classified into six brainwave signals by the signal analysis part 110, and each detected signal is inputted into the LED display part 112, and the data measured by the acceleration sensor is used for the sake of a brainwave-related external instrument control.

The battery part 110 is provided to manage the electric power of all the elements shown in FIG. 1. When the power lacks, a signal is transmitted to the LED display part 112, and the internal battery 114 is charged by the battery charging part 116. The blue tooth transmission part 118 is configured to a blue tooth signal for the sake of a wireless communication with a data receiving system 200.

FIG. 2 shows a data receiving system 200 which receives a signal from the blue tooth transmission part 118 and processes the signal. As shown in FIG. 2, the data receiving system 200 transmits the processed brainwave signal received from the blue tooth receiving part 202 to the input terminal of the MCU 208. In the MCU 208, the LCD 206 and the output part 214 convert electric signals into numeric forms, and the signal from the brainwave measuring and data transmission system is stored in the memory. The PC connection part 216, the instrument control part 218 and the blue tooth 220 enable the brainwave-related external instrument to have a wired or wireless control using the brainwave signal and a data measured by the brainwave signal of the external instrument. The battery part 210 manages the electric power of all the elements shown in FIG. 2, and when power lacks, a signal is transmitted to the LCD 208, so the internal battery 210 is charged by the battery charging part 212.

As shown in FIG. 3, in the portable brainwave measuring and controlling system of the present invention, the brain signal measuring by three headset sensor parts 112 and the data measured by the acceleration sensor are transferred from the blue tooth transmission part 118 to the blue tooth receiving part 202 through the blue tooth. The LCD part 206 allows a user to measure his brainwave in a numeric output form. In addition, the three headset sensor parts 112 comprise batteries 114 needed in each headset. Electric power used in the headset is supplied through the battery 114. The LCD part 206 is configured to display an electric value of the brainwave through the blue tooth receiving part 202 in a numeric form. The power and time setting part 302 helps a user to set a needed measurement time like 1 minute 3 minutes and 5 minutes for thereby displaying in the numeric forms on the brainwave. The connection part 304 provides a communication and control function with a brainwave-related external instrument through a personal computer or a blue tooth. In case of multiple users, the number of measurement persons can be manually or automatically set using the setting switch 306.

As shown in FIG. 4, in the portable brainwave measuring and controlling system, if that the data transmission systems 100 are used in multiple numbers, it is possible to concurrently display multiple signal values on the LCD 206 based on the time division communication method as the MCU 108 analyzes the signals. The blue tooth 220 is used when connecting with a nearby cellular phone, PDA and personal computer.

As shown in FIG. 5, the acceleration sensor 120 is configured to output an acceleration value of a 3-axis direction formed of X, Y and Z-axes in a form of a constant data signal. The acceleration sensor 120 is engaged at a head portion of a human for detecting the motion of the head for thereby controlling the direction of the brainwave-related external instrument.

Here, the acceleration sensor 120 can be engaged at the head of the person in a form of a headset. It can be manufactured in various forms depending on the user's selection and is worn on a head portion.

FIG. 5A shows an operation principle of the acceleration sensor 120 according to a preferred embodiment of the present invention. The direction of the brainwave-related external instrument can be controlled depending on the motions of the head to which the acceleration sensor 120 is attached, i.e. the direction and the inclination of the head.

The acceleration sensor 120 detects the motions in the directions of the X, Y and Z-axes in the 3-dimensional space and outputs it in a form of signals. The X-axis is a leftward and rightward direction, and the right side is a + signal, and the Y-axis is a forward and backward direction, and the forward side is a + signal, and the Z-axis is an upward and downward direction, and the downward side is a + signal.

The acceleration sensor 120 measures the inclination value with respect to the directions of X, Y and Z-axes and determines the direction of the brainwave-related external instrument.

FIG. 5B shows a range of the inclination of the acceleration sensor 120. In the present invention, the moving range of the brainwave-related external instrument is classified into four stages as shown in the following table.

TABLE 1

| | |
|---|---|
| 316°-360° range and 0°-45° range | Forward (forward region) |
| (88), (81) | |
| 46°-135° range (82), (83) | Left turn (leftward side region) |
| 136°-225° range (84), (85) | Backward (backward region) |
| 226°-315° range (86), (87) | Right turn (rightward side region) |

FIG. 6 shows a neutral state of the acceleration sensor 120. It is preferred that a neutral signal is generated by the acceleration sensor 120 during an operation that the head rotates only in the leftward and rightward directions with a user's head not being inclined, which is intended to prevent an erroneous situation that the brainwave-related external instrument moves in the not-intended direction since the user might turn his head in the leftward or rightward direction owing to a surrounding environment or in an unconscious state.

As shown in FIG. 6, the range within the inclination of ±5° in the leftward and rightward directions about the Z-axis is set as the neutral region inclination in the present invention. The above mentioned neutral region inclination is not limited to ±5°, but could be properly adjusted by the user.

The headset type wireless transmission part of the present invention comprises a built-in LED so as to check with the eyes what kind of signals are outputted during the measurement of the brainwaves and the moving direction of the head, so the LEDs corresponding to delta wave δ, theta wave θ, alpha wave α, SMR wave, beta wave β and gamma wave σ, respectively, are turned on and off depending on the kinds of the detected brainwaves for the kinds of the measured brainwaves to be easily recognized. In addition, the data can be transmitted to the wireless receiving part positioned in the local area through the headset type wireless transmission part, and it can be checked on the END or LCD screen so that the brainwave analysis can be conveniently performed by using the data of the brainwaves received through the local area wireless communication, whereby it is possible to easily measure the brainwaves without an additional process system such as a personal computer, etc. The brainwave-related local or remote external instrument can be controlled in a wired or wireless way as it is connected with a wired or wireless communication instrument such as a personal computer or a blue tooth with respect to the brainwave signals and the data measured by the acceleration sensor by way of the connection part.

### ADVANTAGEOUS EFFECTS

The portable brainwave measuring and controlling system according to the present invention makes it possible to perform a user's concentration power measurement and to objectively judge the comparative analysis since the measured values can be displayed and stored in a numeric form. In addition, the user of the present invention can conveniently use the present invention since the received signals are processed in a numeric form for the signals to be displayed from 0 to 100 which is different from the conventional simple work having features in that the wirelessly received brainwave signals are directly adapted to the toy or for the sake of the instrument control in the wireless receiver, and the output setting can be performed based on the numerically processed brainwave data, and the analysis and control can be variously and accurately performed, and the measured data can be more objectively judged.

The present invention is advantageous in performing a complicated control since the signal based on the inclination direction of the head part can be detected by the wireless headset shaped acceleration sensor without using an additional wireless controller for thereby controlling the direction of the brainwave-related external instrument in a wireless form, and the signal detected by the acceleration sensor and the numerically processed brainwave data are concurrently used for thereby concurrently controlling the direction and the speed of the brainwave-related external instrument. In addition, the various internet-based applications are made possible by the compatibility with the personal computer with the aid of the blue tooth module in the interior of the wireless receiver. A wireless communication and control with the local area external instrument are advantageously possible. In addition, the brainwave state and concentration power state of the patient and ordinary user can be measured in real time for 24 hours on the local area measurement or internet in terms of the application of the present invention.

The present invention can be applied to when numerically processing six kinds of brainwaves for the sake of visual checks, not applying to the instrument controls by checking only the alpha or SMR waves which emit in the course of intensive concentrations. As the present invention is combined with the acceleration sensor, the control of the complicated brainwave-related external instrument can be possible, so the present invention has good effects when performing a concentration power enhancement research.

The present invention can be applied to a wireless game apparatus using a brainwave signal, an education learning device, etc. instead of the brainwave measurement.

The present invention has been described along with the limited drawings and embodiments, but the present invention is not limited to the disclosed embodiments, and it is obvious that an ordinary person skilled in the art can perform various modifications and changes from the disclosures.

So, the concepts of the present invention should be interpreted based on the claims, and all the equivalents or other modifications belong to the scope of the present invention.

### BRIEF DESCRIPTION OF DRAWINGS

FIG. 1 is a block diagram of a construction of a measurement value transmission of a brainwave measurement and an acceleration sensor signal of a portable brainwave measuring and controlling system according to the present invention.

FIG. 2 is a block diagram of a construction of an output system and an instrument control system which are configured to receive a brainwave from a transmitter of a portable brainwave measuring and controlling system and a signal measurement value of a signal measurement value of an acceleration sensor according to the present invention.

FIG. 3 is a concept view of a use environment of a portable brainwave measuring and controlling system according to the present invention.

FIG. 4 is a concept view illustrating a use environment in which a plurality of users can concurrently measure with a time division method in a portable brainwave measuring and controlling system according to the present invention.

FIG. 5 is a view of an example for describing an operation principle of an acceleration sensor according to the present invention.

FIG. 6 is a view of an example for describing a neutral state of an acceleration sensor according to the present invention.

FIG. 7 is a view of an experiment measurement data of a frequency-based brainwave output signal in a concentration state.

FIG. 8 is a view of an experiment measurement data of a direction detection signal using an acceleration sensor.

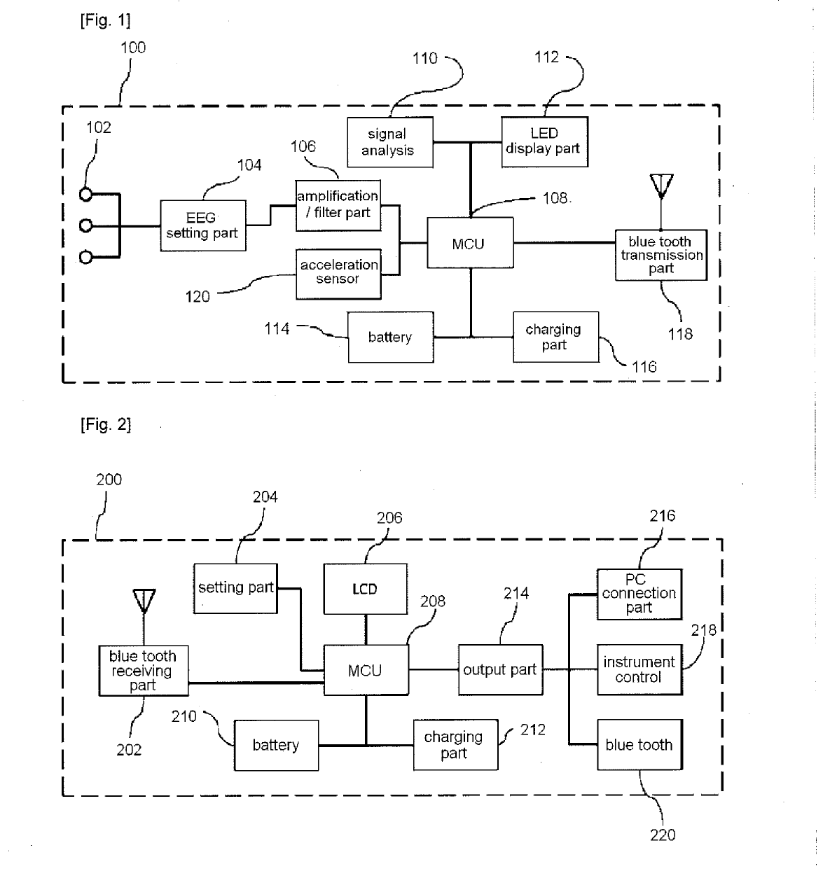

<Descriptions of Key Elements of the Drawings>

| | |
|---|---|
| 100: data transmission system | |
| 102: headset sensor part | 104: EEG input part |
| 106: amplification/filter part | 108: MCU |
| 110: signal analysis part | 112: LED display part |
| 114: battery | 116: charging part |
| 118: blue tooth transmission part | 120: acceleration sensor |
| 200: data receiving system | |
| 202: blue tooth receiving part | 204: setting part |
| 206: LCD | 208: MCU |
| 210: battery | 212: charging part |
| 214: output part | 216: PC connection part |
| 218: instrument control part | 220: blue tooth |
| 302: power and time setting part | 304: connector |
| 306: manual/automatic switch | |

### BEST MODES FOR CARRYING OUT THE INVENTION

The advantages and features of the present invention and the method for achieving the same will be more clear with the references to the embodiments which will be described later; however the present invention is not limited to the following embodiments, but is implemented in various types, and such embodiments might help clarify the disclosures of the present invention, and are provided to inform an ordinary person skilled in the art to know the scope of the invention which can be defined by the claims.

### Embodiment

The embodiment of the present invention is directed to proving a relationship between the alpha wave production ratio and the concentration power by checking the alpha wave which is a brainwave related to when a testee is concentrating and the testee does not concentrate and the production ratio of the brainwave except for the above mentioned state, and the present invention is directed to providing a control method of a wireless electronic instrument using the same.

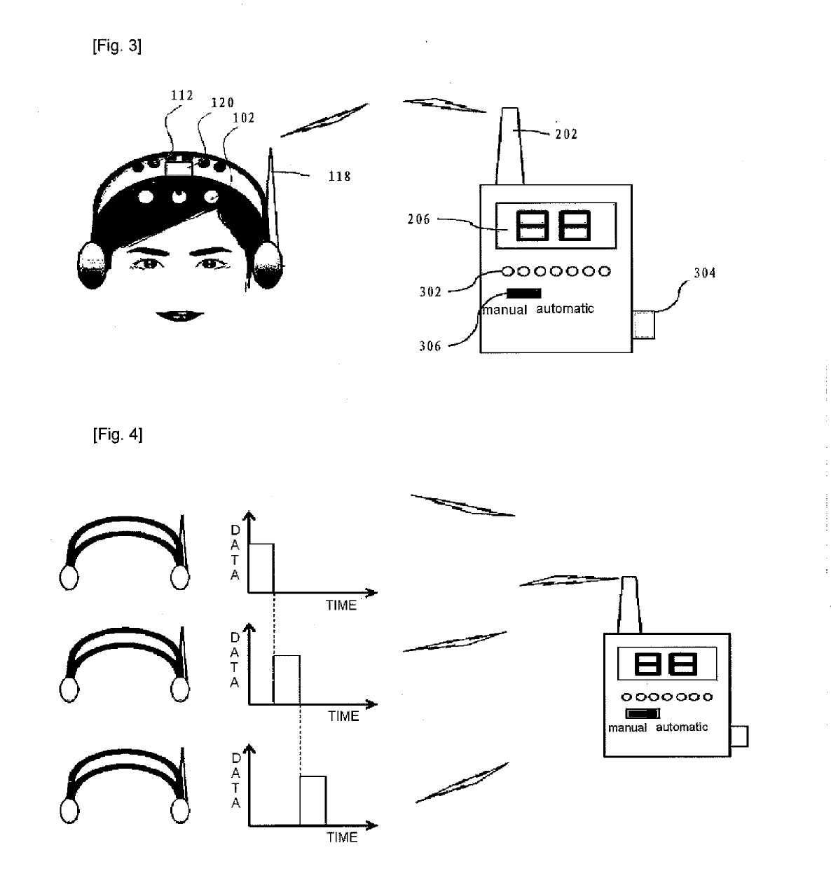

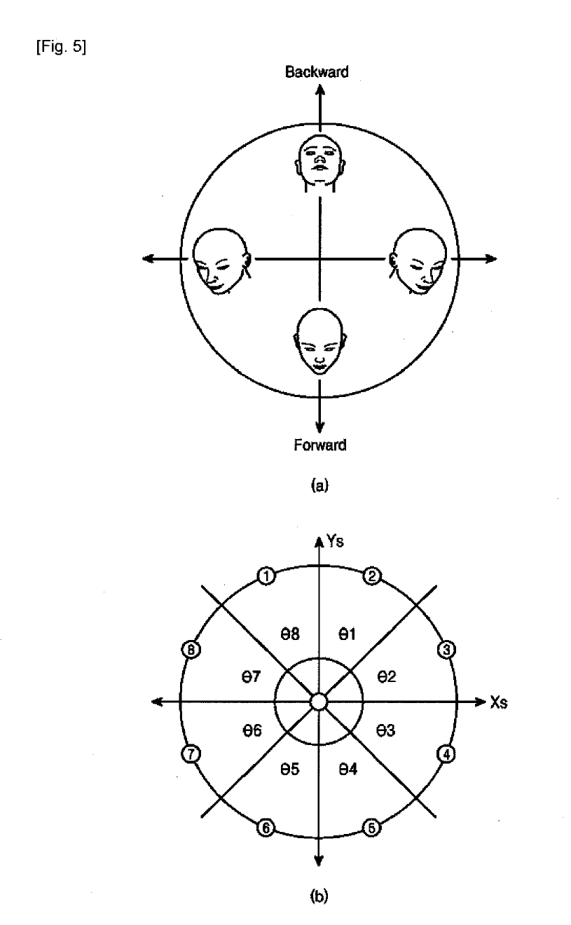

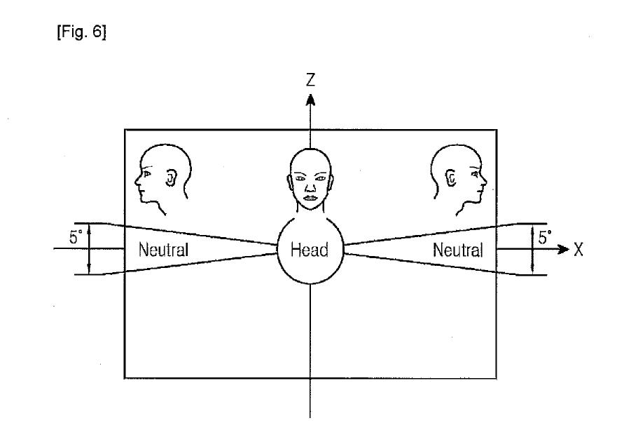

The acceleration sensor is engaged in the interior of the brainwave detection part configured to detect the brainwaves of the headset type which is worn on a testee's head for thereby forming an apparatus which makes it possible to detect the brainwave and the direction from the acceleration sensor (FIG. 3). Since the brainwave signal detected by the brainwave detection part is a weak signal of a couple of micro volts, it is needed to amplify to a level higher enough for the sake of analysis and actual application, and a filter system is needed so as to handle the noises which are inevitably produced. The production ratio of the alpha waves is tested in case of concentration, and the measurement is performed with the testee being assumed to have two concentration states of meditation and reading.

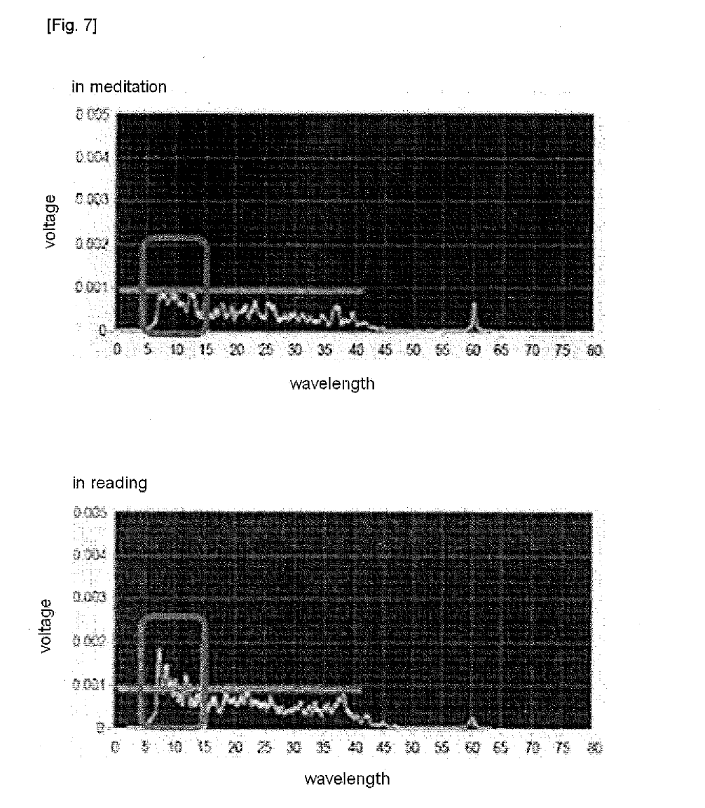

As a result of the test, in case of the concentration such as reading and meditation, the production ratio of the alpha wave is higher than the brainwave (beta wave and gamma wave) excluding the alpha wave, and in case of the reading, the production of the alpha wave two times higher than the meditation is obtained. So, it has been confirmed that the relationship between the alpha wave and the concentration power can be proved in the present embodiment (FIG. 7).

In the next stage, since the production ratio of the alpha wave is numerically processed by the instruments of the present invention, the state of the concentration power is converted for the same of visual checks. In addition, the acceleration sensor provided in the interior of the brainwave detection part is configured to calculate the coordinate values with respect to the movements on the X, Y and Z-axes. The X-axis is the leftward and rightward directions, and the rightward side is a + signal, and the Y-axis is the forward and backward direction, and the frontward side is a + signal, and the Z-axis is the upward and downward direction, and the downward side is a + signal. So as to check the coincidence between the motion of the head to which the acceleration sensor of the testee is attached and the coordinate value, the present invention is directed to dividing the scope based on the inclination of the acceleration sensor into the movements of the forward side (X-axis, + value), the backward side (X-axis, − value), the leftward side (Y-axis, + value), and the rightward side (Y-axis, − value). The coincidence as compared to the coordinate value which is actually measured is checked.

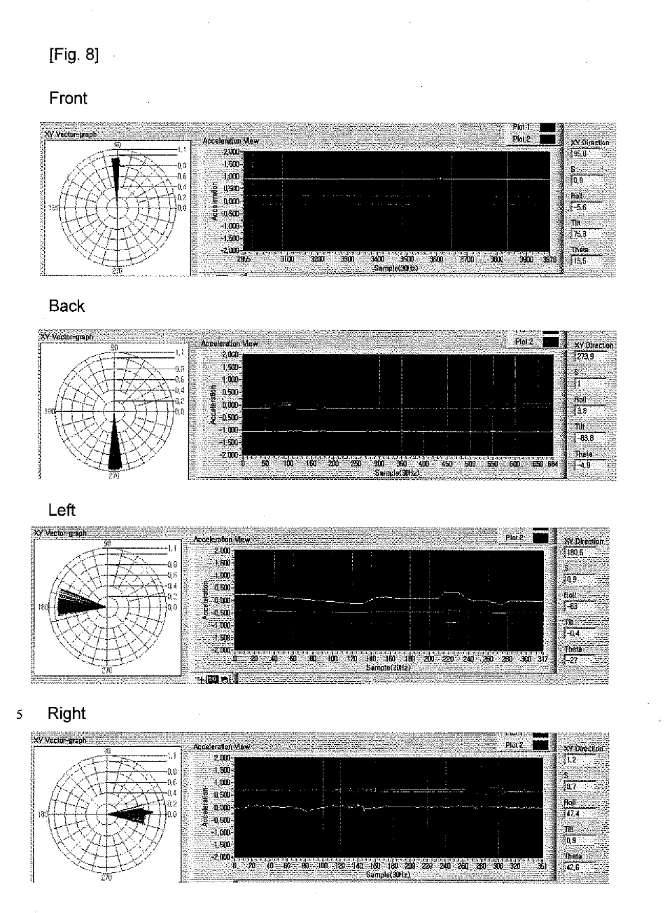

(FIG. 8)

As described above, the direction can be detected using the coordinate value inputted from the acceleration sensor. The brainwave measuring and controlling system using the alpha wave with a reliability which is the key point of the present development can be implemented by concurrently using two kinds of the input signals. Since the alpha wave and the direction signal are concurrently applied, it is also possible to implement a control of the wireless electronic instrument with the aid of concentration power.

### INDUSTRIAL APPLICABILITY

The portable brainwave measuring and controlling system according to the present invention can be widely applied to the fields of an electronic instrument control using a brainwave. For example, it is possible to control a relatively simple instrument such as an electric fan control, a lamp control, etc. The television On/Off control and the channel selection are possible as well by analyzing and processing the brainwave algorithm. In case of a wireless motored train, it can be controlled to move forwards or to stop at a certain position and can be controlled to move backwards.

The production ratio of the alpha wave related to the concentration powder can be numerically processed with the units of 0-100, and it can be applied to the class which plans to enhance concentration power.

When the present invention is applied to the driving of a vehicle, it can be well applied to a system which can protect the health of the driver by checking the patterns of the brainwaves changing in terms of a driving for an emergency protection, a dozing driving or a driver's health state.

Therefore, the present invention can be well applied to various fields such as an electronic instrument control, a concentration power enhancement, a wireless brainwave toy control, etc.

1. A portable brainwave measuring and controlling system, comprising:
a LED output part which is formed of a filter part configured in such a way that when an analog brainwave signal is measured by a brainwave measuring part configure to measure a wave by attaching a plurality of electrodes to a testees head skin, the noises contained in the analog brainwave signal measured by the brainwave measuring part are removed, and a MCU part configured to control the whole portions of the portable brainwave measuring system processes the signals passed through an amplification part amplifying the brainwave signals to six kinds of brainwaves such as delta wave δ, theta wave θ, alpha wave α, SMR wave, beta wave β and gamma wave σ, and each output signal is displayed as a LED light;
a headset type wireless transmission and receiving part which wirelessly transmits the six kinds of brainwaves;
a display part which numerically displays the values outputted following the process by the MCU in a form of 0-100 with the aid of a LCD apparatus;
a memory part storing at least one output value; and
a connection part which makes possible a wired or wireless connection and control with a brainwave-related external instrument by using a personal computer and a blue tooth with the aid of an output value of an acceleration sensor outputting an acceleration value of 3-directions formed of X, Y and Z-axes in a form of constant data and a brainwave value outputted over the process of the MCU.

2. The system of claim 1, wherein the display part using the LCD apparatus comprises:
an output display apparatus which numerically expresses the value of the brainwave analyzed over the process of the MCU in a form of 0-100 by the six brainwave signals, so a user can compare and analyze in a numeric form for thereby obtaining an objective data for the sake of a concentration power enhancement.

3. The system of claim 1, wherein the connection part comprises:
an apparatus which can be remotely monitored by adapting it to a personal computer, etc. and has a compatibility with a brainwave-related external instrument with the aid of a personal computer, a blue tooth and a RF signal since a wireless module can be used when a connection is impossible with a wired connection.

4. The system of claim 1, further comprising an apparatus which accurately controls a brainwave-related external instrument based on the classified outputs in a corresponding range by determining the ranges of the indication value like a 1st output when the indication value of the measured brainwave on the display part is 0-10, a 2nd output in case of 11-20, and a 3rd output in case of 21-30.

5. The system of claim 1, wherein the output part comprises a display means which displays what kinds of waves are being displayed by flashing the LEDs corresponding to each signal by classifying the brainwaves measured by the brainwave measuring part into six brainwave signals depending on frequencies.

6. The system of claim 1, further comprising a system which helps obtain a concentration power measurement and helps use a measured signal since the measured brainwave can be objectively compared and analyzed by dividing and numerically processing the signals from 1 to 100 with the aid of the number that the wirelessly transmitted brainwave signals are inputted per hour.

7. The system of claim 1, wherein a complicated control of the brainwave-related external instrument is possible with an EEG and acceleration sensor data as the MCU analyzes two signals detected by the EEG (brainwave signal) and the acceleration sensor.

8. The system of claim 1, further comprising:
a wireless receiving apparatus which makes it possible to obtain a time-based value of a to-be-measured brainwave by enabling the wireless receiver to set a brainwave measurement time and the kinds of the brainwaves (1 minutes-10 minutes) and by numerically displaying the value of the received and to-be-measured brainwave for 1 minute in case of the 1-minute setting and the value of the received and to-be-measured brainwave for 3 minutes in case of the 3-minute setting.

9. The system of claim 1, further comprising:
a wireless transmission and receiving apparatus which includes a built-in blue tooth module, RF module and acceleration sensor in the interior of a headset type wireless transmitter or wireless receiver for the sake of a communication between the personal computer and the wireless instruments.

10. The system of claim 1, wherein in the interior of the wireless transmitter worn on a user's head skin is provided an acceleration sensor, so the brainwave measurement and the user's head motion signal are concurrently recognized for thereby controlling the instrument by concurrently using the concentration signal and the motion signal.

11. The system of claim 1, wherein the display part comprises:
a display apparatus which is configured to manage a plurality of brainwave measurement users since a plurality of wireless receivers can be managed with one wireless transmitter by measuring multiple brainwaves used within a local area, not displaying only one wireless receiver.

12. The system of claim 11, wherein the display apparatus comprises another display apparatus which makes it possible to check errors in the portable brainwave measuring apparatus which shows the kinds of the set brainwaves and the received brainwave values and shows the lack of the battery of the instrument and the errors of the instrument.

13. The system of claim 1, wherein the memory part comprises an apparatus having a function of storing a result value obtained by analyzing the value of the brainwave received from the portable brainwave measurement apparatus over the process of the MCU part by the number determined by a user's setting.
